SEQUENCE LISTING (8 sequences)

SEQ ID NO: 1 - DNA, 501 bp, Homo sapiens
  misc_feature 425: SNP4 G/T
ccaccgacctcctcccggggccccaggtaggacagcctggatgctgggcaccgagccaggggcctcggggctgggggcagcttgggtgctagcgagaggaagttcctgggggcgggcatgcggggagacacagcacccctgtggggggggcttctttctccaggctgggccttggcctccagacgcctctctgcccaccaacccctttgtattgagaggggcctcgacccctcctcatcccctcagtgaaaacaataaagacaaatttttgttgcttcagtgacttctgtgggcctgcgggcagggagggaccagtccatggaacgggaagtgggaccctgtagtttctgctgtgagagaaaaagacctcagctgttgggggtcctcccctgcctcccctggcaacccatccaccttcccccackccacccacacatggccttgtgggtctggccctccaccccgtggtagagacacagatacgggtcctggagaagacac

SEQ ID NO: 2 - DNA, 401 bp, Homo sapiens
  misc_feature 201: SNP5 A/G
ctgggcaagatggtgcctacaggcagatatgaaacaggtgggctggcacctgggcacagtgcttgcccctgctgcctcttccctcccaggtatgggaaaagacacaaagaggacacgctggccttctcggagtgggggtccccgcatgctgctgtccccaggtgagtttgactccctgccctgtctgtccagctccctggrgctgaaatgggggtggtgggactgaatcagggcttggaaaggtgtagtggggggtggaagagggagaacaggagcccagggccagcctgaggcctcctgagggcacgaggcctaccccctacactgccatgttctgccctgtcctcacagggagctcagcccgctggacttataatgccaccttctgtctcctacgactc

SEQ ID NO: 3 - DNA, 401 bp, Homo sapiens
  misc_feature 201: SNP6 A/G
ttcccctgcccaacaccacgccctctggcacaaggtggatggccactggcagtgatggggatggagtaagtgctatgagactaggagaggaacaccccaggaacacctgaggtcacagagtgaatgaaggcaggtaaaggagcaaagggaatggggggcccccacatacgtgaccccagggcagcagcttggcctgcaccragtcctccatcacacacactcccccgccccacccccgcatcctggctgtctaaccacaatccctgctgccttcagttaatcaaccaatgagtggtcattcattgcacatctgttctgggcctcatgggggcagcgggaggtgatgaagaagcaagggttctacaaagaggtcaaagctacgtgaggagccggggaatggc

SEQ ID NO: 4 - DNA, 1912 bp, Homo sapiens
  misc_feature 201: SNP7 C/T
ttggtggcaattgtttgtcattacaacctggggcgcaaagtggaatccctgtcctctatccaccgaggacccctggggtattttggcggctggacctgcgccctgagtccaggaggctcgatattcacccttaggatattttcctgaaccccgataccccacaatctcctatccgcacattaactttaccttgggatatgyaaatattaagtagaaccctatgaaatttacattagttaaccgtttttgtttggtttggtttggtttggttatgtttgagacagagtctcgctctgtcacccacgctggaggcgatctcagctcactgcaacctccgcctcccgggttcaagtgattctcctgcctcagcctcccgagtagctgggaccacagatggttataacaatacctcccatcccacatgcccttccacaaggaggccctgacatctcccatcaagaaatgcagacgggccgggcacggtggctcacgcctgtaatcccagtgctttgggaggcccaggtgggtggatcacttgaggtcaggagttcaagaccagcctgaccaacatggtgaaaccccgtctctactaaaaatacaaaaattagccgggtgtggtggcaggcgcctgtagtcccagctacttgggaggctgaggcaagaggattgcttgaacccaggaggcagaggttgcggtgagctgagcagccactgcactccagcctgggtgacactccatctcaatgaaaaaaaaaaaaaaaaacgaaaagaagagaaatgcaggcatgcaggcggcgacagtggctcacgcctgtaatcccaacactttgggaggccaaggcagttggatcgcttgagctcaggagtttaagactagctgggcaacatagcaaaactctgtctctacaaaaaaagaaagaaaaaaattatatatatatatgaaagaaatgcagcccatgacttcccttgagtctggtgggcttgtgacacacttataaccaataggatgggtcagaagtaatgctacaggactgctaaggttgaactttaaaagacaatgcagcagccaccttttctgctggaccaggtgggcttgaagcctccagctgccctgtaagcagcccaactgccctgaagctgctccaaatcagcctgcccagagagaccactctgagactacgtggagagagagagagagatgcttgtccagccccctggacccaggccccacccttgctgtcccagctccagccatcatctgactgcaaccacatgagaaactctgaactagaaccaccaggtaagcccttcctgaattcctcatggagagcatccatgagcaaataaaatggttctttcaagccatggggttttggggtaattttcattttttgagacagtgtctcactctgtcacccaggctggagtgcagtggtgtaatctcagcccactgcaacctccacctcctggcaggtgatcctcctacctcagcctcccaagcagctgggaccacatatgtccaccaccagacctggctaatttcttgttttttatttttactttatgctgggtttttttgtttttgttggtttgtttatttgttcatttgttttgagacagggtcttgctctgttgcccaagctggagtgcagtggcgcaatctcagctcactgcaacccccacctcctgggcttaggccatcctcccacctcagccacccgagtaggaactacaggcgcacaccaccacgcccagctaatttttgtatttttttatagagacaaggttttgccatgttgcccaggctggtctcaaattcctgagctcaagcaatgtacctgcctcagcctcccaaagtgctgggattacaggtatgagccactgcacgtggcctaattttttacagtttctt

SEQ ID NO: 5 - DNA, 401 bp, Homo sapiens
  misc_feature 321: n = a, t, g or c
gccttgccctaaaaactgtgtcctacatcacctccccacacccaggtacttcaaacacagtgatggcagcttctccaacagcagtggatcttcttctggtttggcctggctgatatccgggactcctatgagttggtcaaccacgccaagggactgccagactcctttcacaagccagcttctgacccaggcagctgaccytcaccatggacactacaggccggggggtggccagggtggaccaaaagccatgccagctgggcatgaccccaggcagccagccacaggctgaagggggcttgttggctgagtgatctgcanaggagaaagcagccccagctctgcccagaggaggcgctgaagtgggacaagcacaggaaagaaggggaccagtctaggac

SEQ ID NO: 6 - DNA, 201 bp, Homo sapiens
  misc_feature 101: SNP9 = C/T
aggtgggggcggggcaggaggcttggctccctcccttccctctgcgccctgactcacctctgtggctccactgcctcctcctccttcctattagcttgacyggccagctccagcgcccgagcctccctctgggtgatgttgtgtttccagctgggaagataaagattagagagtgggcaagctgcggagactccagtctag

SEQ ID NO: 7 - DNA, 1212 bp, Homo sapiens
  misc_feature 1012: SNP10 = C/T
gacacctctctagataatgtgatgggggcattgccacatcaccaaagccataggccactgtccagagctgggattcagatgactgtggacctactctctcccacccagaagtagcctgttgccttgtgggagtgtccccctgccagttagtgccaccctaccccaagtctgtcttcactacaagtacccttttttatttttatttttattttttgagacagagtcttgttttgtcacccaggctggagtgcagtggcacgatcttggctcactacaacctctgcctccaggtctgggttcaagcgattctcctgcctcagcctcccaagtagctgggactacaggcacgtgccaccatgcctggctaatttttgtttgttttttttttttttgtttgtttgtttgttttgagatggagtctcgctccgtcgcccaggctggcaggcagtggcgccatctcggctcactgcaacctccgcctgccgggttcacgccattctcctgcctcagcctcccgagtagctgggactacaggcacctgccaacatgcccggctaattttttgtgtttttagtacagacggggtttcactgtgttagccaggatggtctcgatctcgtgacctcgtgatccacccgcctcggcctcccaaagtgctgggattacaggcgtgagtcactgcgcccggccccctttcttttttcttttctttttttcttttttgagagagtctctctttgttgcccatgctggagtgcagtgaactcgatcttggctcactgtaacttctgcctcccaggttcaagtgattctcttgcctcaacctcccaagtagctgggattacagccgtgcacgaccatgcccagctaatttttgtatttttagtagagactgggtttcaccatgttggccaggctggtctcgaacccctgacctcaggtgatccgcccgtgtcgtcctcccaagcgctgggattacagacgtgattcatcgcgcctggcctacaagtaycccttctaactgagggcaactgtaaaggaagtagggcatagaagaatagtagtgggccctgtgcccagaagggaggaggaggaagaaaaaagggggtacttagggtaccctacccacaggcaggccccagacagcagctggagatagccaaatgttaatcaccaattagcacagttcaggtggaaagggcaactctatta

SEQ ID NO: 8 - DNA, 1000 bp, Homo sapiens
  misc_feature (41)..(140): n = a, t, g, or c
gcccacctcagcctcccaaagtgctgggattacaggcgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatggctttggtgatgtgcaatgcccccatcacattatctagagaggtgtcccaagggctcaatgttgagggaatatgaaggatcggggtttcctagggacagctggataaactgagaacatacattcaagctgtcttcctgctcaggggcaggataaagcaggagaaagagacatgtttgtacctcaggggctcacaggagggtggggtagttacaactttatctatagagatttaggagcccctctcctcatatgtcctactgtcctgtacaagagaagtatgtcatcctgtctcttctatcagagtgaagaccctggagttacgctgtgcctctctattatacaggagccactccarggcaggggctttctgtccctgatctgcctgggagctctctgagaggaggaggagagctggaggcgatgttcccccagcacactgaggctttctgaggctaagggctgtgtctccccctcagactggagactccttgggatactccctttgcctttctccaagtacttagcacatggctctgtccacaggggctctcagtctagaaggggcagggcacagattgcatttttggcagacctccaaggcgttctttccaggctcctcctctgccaccgccatgggtacttgggaccataaacagccaagctcatgctctgcctatcagctccaggtcctaataaaggccaccttcctttccaggtgaacaccctagcctaggagcagatccagcatgaaattaagtgtgcgtgtgggatgagaggtacctggactagcagggtcacttacctctgcctcctggaaacctcctctgcatacccacccattctccaccatctaga

(19) United States
(12) Patent Application Publication
(10) Pub. No.: US 2008/0254450 A1
(43) Pub. Date: Oct. 16, 2008

(54) Human Obesity Susceptibility Genes Encoding Peptide Hormones and Uses Thereof

(76) Inventors: Anne Philippi, St Fargeau Ponthierry (FR); Francis Rousseau, Savigny sur Orge (FR)

Correspondence Address: FISH & RICHARDSON PC, P.O. BOX 1022, MINNEAPOLIS, MN, 55440-1022, US

(21) Appl. No.: 10/593,414

(22) PCT Filed: Mar. 23, 2005
(86) PCT No.: PCT/IB05/01030

§ 371 Date: Sep. 18, 2006

Related U.S. Application Data
(60) Provisional application No. 60/555,700, filed on Mar. 24, 2004.

(51) Int. Cl. C12Q 1/68

(52) U.S. Cl. 435/6

(57) ABSTRACT

The present invention discloses the identification of human obesity susceptibility genes, which can be used for the diagnosis and prevention of obesity and related disorders. The invention more specifically discloses that the PPY and PYY gene on chromosome 17 and certain alleles thereof are related to susceptibility to obesity. The present invention relates to particular mutations in the PPY and PYY gene and expression products, as well as to diagnostic tools and kits based on these mutations. The invention can be used in the diagnosis of predisposition to or protection from, detection, prevention and/or treatment of coronary heart disease and metabolic disorders, including hypoalphalipoproteinemia, familial combined hyperlipidemia, insulin resistant syndrome X or multiple metabolic disorder, coronary artery disease, diabetes and dyslipidemia.

### FIELD OF THE INVENTION

The present invention relates generally to the fields of genetics and medicine.

### BACKGROUND OF THE INVENTION

Approximately three to eight percent of the total health costs of modern industrialized countries are currently due to the direct costs of obesity (Wolf, 1996). In Germany, the total costs (both direct and indirect) related to obesity and comorbid disorders were estimated at 21 billion German marks (29.4 US Dollar) in 1995 (Schneider, 1996). By 2030 these costs will rise by 50% even if the prevalence of obesity does not increase further.

Obesity is often defined simply as a condition of abnormal or excessive fat accumulation in adipose tissue, to the extent that health may be impaired. The underlying disease is the process of undesirable positive energy balance and weight gain. An abdominal fat distribution is associated with higher health risks than a gynoid fat distribution.

The body mass index (BMI; kg/m²) provides the most useful, albeit crude, population-level measure of obesity. It can be used to estimate the prevalence of obesity within a population and the risks associated with it. However, BMI does not account for body composition or body fat distribution (WHO, 1998).

TABLE 1
Classification of overweight in adults according to BMI (WHO, 1998)

| Classification | BMI (kg/m²) | Risk of co-morbidities |
|---|---|---|
| Underweight | <18.5 | Low (but risks of other |
| | | clinical problems increased) |
| Normal range | 18.5-24.9 | Average |
| Overweight | ≧25 | |
| Pre-obese | 25-29.9 | Increased |
| Obese class I | 30-34.9 | Moderate |
| Obese class II | 35-39.9 | Severe |
| Obese class III | ≧40 | Very severe |

Obesity has also been defined using the 85^{th} and 95^{th} BMI-percentiles as cutoffs for definition of obesity and severe obesity. BMI-percentiles have been calculated within several populations; centiles for the German population based on the German National Nutrition Survey have been available since 1994 (Hebebrand et al., 1994, 1996). Because the WHO classification of the different weight classes can only be applied to adults, it has become costumary to refer to BMI-percentiles for the definition of obesity in children and adolescents.

The recent rise in the prevalence of obesity is an issue of major concern for the health systems of several countries. According to reports of the Center of Disease Control and Prevention (CDC) there has been a dramatic increase in obesity in the United States during the past 20 years. In 1985 only a few states were participating in CDC's Behavioral Risk Factor Surveillance System (BRFSS) and providing obesity data. In 1991, four states were reporting obesity prevalence rates of 15-19 percent and no states reported rates at or above 20 percent. In 2002, 20 states have obesity prevalence rates of 15-19 percent; 29 states have rates of 20-24 percent; and one state reports a rate over 25 percent. Similar trends have been observed in other countries in Europe and South America.

Children and adolescents have not been exempt from this trend. Quite to the contrary, the increase in the USA has been substantial. Thus, between the 1960ies and 1990, overweight and obesity increased dramatically in 6 through to 17 year olds. The increments translate into relative increases of 40% using the 85^{th} BMI-centile (calculated in the 1960ies) as a cutoff and 100% upon use of the 95^{th} centile. In a cross sectional study of German children and adolescents treated as inpatients for extreme obesity between 1985 and 1995, a significant increase of the mean BMI of almost 2 kg/m² over this ten year period has been reported. Within this extreme group, the increments were most pronounced in the uppermost BMI ranges.

The mechanisms underlying this increase in the prevalence of obesity are unknown. Environmental factors have commonly been invoked as the underlying cause. Basically, both an increased caloric intake and a reduced level of physical activity have been discussed. In England the increase in obesity rates has been attributed to the latter mechanism. Thus, in this country, the average caloric intake even decreased somewhat within the last two decades, whereas indirect evidence stemming from the increases in hours spent watching television and from the average number of cars per household points to reduced levels of physical activity as the relevant causative factor.

Genetic factors have previously not been considered as a contributing cause. Quite to the contrary, the fact that the increased rates of obesity have been observed within the last two decades has been viewed as evidence that genetic factors cannot be held responsible. However, it has been proposed that an increase in the rate of assortative mating could very well constitute a genetic contribution to the observed phenomenon. This hypothesis is based on evidence suggesting that stigmatisation of obese individuals represents a rather recent social phenomenon, thus invariably having led to increased rates of assortative mating. As a consequence, the offspring have a higher loading with both additive and non-additive genetic factors underlying obesity. Indeed, an exceedingly high rate of (deduced) assortative mating amongst the parents of extremely obese children and adolescents has been observed.

Potentially life-threatening, chronic health problems associated with obesity fall into four main areas: 1) cardiovascular problems, including hypertension, chronic heart disease and stroke, 2) conditions associated with insulin resistance, namely Non-Insulin Dependent Diabetes Mellitus (NIDDM), 3) certain types of cancers, mainly the hormonally related and large-bowel cancers, and 4) gallbladder disease. Other problems associated with obesity include respiratory difficulties, chronic musculo-skeletal problems, skin problems and infertility (WHO, 1998).

The main currently available strategies for treating these disorders include dietary restriction, increments in physical activity, pharmacological and surgical approaches. In adults, long term weight loss is exceptional using conservative interventions. Present pharmacological interventions typically induce a weight loss of between five and fifteen kilograms; if the medication is discontinued, renewed weight gain ensues. Surgical treatments are comparatively successful and are reserved for patients with extreme obesity and/or with serious medical complications.

Recently, a 10 year old massively obese girl, in whom a leptin deficiency mutation had been detected, was treated successfully with recombinant leptin. This is the first individual who therapeutically profited from the detection of the mutation underlying her morbid obesity.

Several twin studies have been performed to estimate heritability of the BMI, some of which have encompassed over 1000 twin pairs. The results have been very consistent: The intrapair correlations among monozygotic twins were typically between 0.6 and 0.8, independent of age and gender. In one study, the correlations for monozygotic and dizygotic twins were basically the same, independent of whether the twins had been reared apart or together. Heritability of the BMI was estimated at 0.7; non-shared environmental factors explained the remaining 30% of the variance. Surprisingly, shared environmental factors did not explain a substantial proportion of the variance. Both hypercaloric and hypocaloric alimentation lead to similar degrees of weight gain or loss among both members of monozygotic twin pairs, indicating that genetic factors regulate the effect of environmentally induced variation of energy availability on body weight. Metabolic reactions and changes in body fat distribution upon overeating and undereating are also under genetic control (reviewed in Hebebrand et al., 1998).

A large adoption study has revealed that the BMI of adoptees is correlated with that of their biological parents and not with the BMI of the adoptive parents. Depending on the family study, the correlation between the BMI of sibs is between 0.2 and 0.4. Parent-offspring correlations are typically slightly lower. Segregation analyses have repeatedly suggested a major recessive gene effect. Based on these analyses, sample size calculations have been performed based on both concordant and discordant approaches.

In contrast to the expectations, the concordant sib-pair approach was superior; a lower number of families were required to achieve the same power.

Family studies based on extremely obese young index patients, either mother or father or both, have a BMI >90^{th} decile in the vast majority of the families. Based on index patients with a BMI >95^{th} centile, approximately 20% of the respective families have a sib with a BMI >90^{th} centile.

In conclusion, it is apparent that environmental factors interact with specific genotypes rendering an individual more or less susceptible to the development of obesity. Furthermore, despite the fact that major genes have been detected, it is necessary to consider that the spectrum reaches from such major genes to genes with an only minor influence.

The discovery of the leptin gene at the end of 1994 (Zhang et al., 1994) has been followed by a virtual explosion of scientific efforts to uncover the regulatory systems underlying appetite and weight regulation. It is currently the fastest growing biomedical field. This upswing has also resulted in large scaled molecular genetic activities which, due to obvious clinical interest, are basically all related to obesity in humans, rodents and other mammals (Hebebrand et al., 1998).

In this respect, many genes in which mutations lead to the presently known monogenic forms of obesity have been cloned in rodents. Systemic consequences of these mutations are currently being analysed. These models have provided insights into the complex regulatory systems involved in body weight regulation, the best known of which includes leptin and its receptor.

In mice, but also in pigs, over 15 quantitative trait loci (QTL) have been identified that are most likely relevant in weight regulation (Chagnon et al., 2003).

In humans, four exceedingly rare autosomal recessive forms of obesity have been described as of 1997. Mutations in the genes encoding for leptin, leptin receptor, prohormone convertase 1 and pro-opiomelanocortin (POMC) have been shown to cause massive obesity of an early onset type, associated with hyperphagia. Distinct additional clinical (e.g. red hair, primary amenorrhea) and/or endocrinological abnormalities (e.g. markedly altered serum leptin levels, lack of ACTH secretion) pinpointed to the respective candidate genes. Both the monogenic animal models and the human monogenic forms have led to new insights into the complex system underlying body weight regulation.

Very recently, the first autosomal dominant form of obesity was described in humans. Two different mutations within the melanocortin-4 receptor gene (MC4R) were observed to lead to extreme obesity in probands heterozygous for these variants. In contrast to the aforementioned findings, these mutations do not implicate readily obvious phenotypic abnormalities other than extreme obesity (Vaisse et al., 1998; Yeo et al., 1998). Interestingly, both groups detected the mutations by systematic screens in relatively small study groups (n=63 and n=43).

Hinney et al. (1999) screened the MC4R in a total of 492 obese children and adolescents. All in all, four individuals with two different mutations leading to haplo-insufficiency were detected. One was identical to that previously observed by Yeo et al. (1998). The other mutation, which was detected in three individuals, induced a stop mutation in the extracellular domain of the receptor. Approximately one percent of extremely obese individuals harbour haplo-insufficiency mutations in the MC4R. In addition to the two forms of haplo-insufficiency, Hinney et al. (1999) also detected additional mutations leading to both conservative and non-conservative amino acid exchanges. Interestingly, these mutations were mainly observed in the obese study group. The functional implications of these mutations are currently unknown.

The identification of individuals with MC4R mutations is interesting in the light of possible pharmacological interventions. Thus, intranasal application of adrenocorticotropin₄₋₁₀ (ACTH₄₋₁₀), representing a core sequence of all melanocortins, resulted in reduced weight, body fat mass and plasma leptin concentrations in healthy controls. The question arises as to how mutation carriers would react to this treatment, which could theoretically counterbalance their reduced receptor density.

The involvement of specific genes in weight regulation is further substantiated by data obtained from transgenic mice. For example, MC4R deficient mice develop early onset obesity (Huszar et al., 1997).

Different groups are conducting genome scans related to obesity or dependent phenotypes (BMI, leptin levels, fat mass, etc.). This approach appears very promising, because it is both systematic and model free. In addition, it has already been shown to be exceptionally successful. Thus, positive linkage results have been obtained even by analysing comparatively small study groups. More important, some findings have already been replicated. Each of the following regions has been identified by at least two independent groups: chromosome 1p32, chromosome 2p21, chromosome 6p21, chromosome 10 and chromosome 20q13 (Chagnon et al., 2003).

### SUMMARY OF THE INVENTION

The present invention now discloses the identification of three human obesity susceptibility genes, which can be used for the diagnosis, and prevention of obesity and related disorders. The invention more specifically demonstrates that the PPY, PYY and/or GIP genes on chromosome 17 and certain alleles thereof are related to susceptibility to obesity.

More particularly, the invention concerns several haplotypes and SNPs that are located in a chromosomal region on chromosome 17 including the PPY, PYY and GIP genes. Preferably, said haplotypes associated with obesity are comprised of SNPs selected from the group consisting of SNP4, SNP5, SNP6, SNP7, SNP8, SNP9, SNP10 and SNP11. In a particular embodiment, said haplotypes associated with obesity are comprised of SNP4, SNP5, SNP6, SNP7 and SNP8. In another particular embodiment, said haplotypes associated with obesity are comprised of SNP9, SNP10 and SNP11. In addition, said single SNPs each independently associated with obesity are SNP7 and SNP8. In a further embodiment, said single SNPs each independently associated with obesity are also SNP9, SNP10 and SNP11.

The invention can be used in the diagnosis of predisposition to or protection from, detection, and/or prevention of obesity, coronary heart disease and metabolic disorders, including, but not limited to, hypoalphalipoproteinemia, familial combined hyperlipidemia, insulin resistant syndrome X or multiple metabolic disorder, coronary artery disease, diabetes and associated complications and dyslipidemia.

A particular object of this invention resides in a method of detecting the presence of or predisposition to obesity or an associated disorder in a subject, the method comprising detecting the presence of an alteration in a gene locus selected from the group consisting of PPY gene locus, PYY gene locus, GIP gene locus and a combination thereof in a sample from the subject, the presence of said alteration being indicative of the presence of or the predisposition to obesity or an associated disorder. In a particular embodiment, the method comprises detecting the presence of an alteration in the PPY and/or PYY gene locus in a sample from the subject. In another particular embodiment, the method comprises detecting the presence of an alteration in the GIP gene locus in a sample from the subject.

An additional particular object of this invention resides in a method of detecting the protection from obesity or an associated disorder in a subject, the method comprising detecting the presence of an alteration in a gene locus selected from the group consisting of PPY gene locus, PYY gene locus, GIP gene locus and a combination thereof in a sample from the subject, the presence of said alteration being indicative of the protection from obesity or an associated disorder. In a particular embodiment, the method comprises detecting the presence of an alteration in the PPY and/or PYY gene locus in a sample from the subject. In another particular embodiment, the method comprises detecting the presence of an alteration in the GIP gene locus in a sample from the subject.

Another particular object of this invention resides in a method of assessing the response of a subject to a treatment of obesity or an associated disorder, the method comprising detecting the presence of an alteration in a gene locus selected from the group consisting of PPY gene locus, PYY gene locus, GIP gene locus and a combination thereof in a sample from the subject, the presence of said alteration being indicative of a particular response to said treatment. In a particular embodiment, the method comprises detecting the presence of an alteration in the PPY and/or PYY gene locus in a sample from the subject. In another particular embodiment, the method comprises detecting the presence of an alteration in the GIP gene locus in a sample from the subject.

This invention also relates to a method for preventing obesity or an associated disorder in a subject, comprising detecting the presence of an alteration in a gene locus selected from the group consisting of PPY gene locus, PYY gene locus, GIP gene locus and a combination thereof in a sample from the subject, the presence of said alteration being indicative of the predisposition to obesity or an associated disorder; and, administering a prophylactic treatment against obesity or an associated disorder. In a particular embodiment, the method comprises detecting the presence of an alteration in the PPY and/or PYY gene locus in a sample from the subject. In another particular embodiment, the method comprises detecting the presence of an alteration in the PPY and/or PYY gene locus in a sample from the subject. In another particular embodiment, the method comprises detecting the presence of an alteration in the GIP gene locus in a sample from the subject.

In a preferred embodiment, said alteration is one or several SNP(s) or a haplotype of SNPs associated with obesity. More preferably, said haplotype associated with obesity comprises SNPs selected from the group consisting of SNP4, SNP5, SNP6, SNP7, SNP8, SNP9, SNP10 and SNP11. In a particular embodiment, said haplotype associated with obesity comprises or consists of SNP4, SNP5 and SNP6 (haplotype 4-5-6). Optionally, the haplotype 4-5-6 can further comprise SNP7 and SNP8. In an alternative embodiment, said haplotype associated with obesity comprises or consists of SNP5, SNP6 and SNP7 (haplotype 5-6-7). Optionally, the haplotype 5-6-7 can further comprise SNP4 and/or SNP8. In an additional embodiment, said haplotype associated with obesity comprises or consists of SNP9 and SNP10, or SNP10 and SNP11. Optionally, said haplotype associated with obesity comprises or consists of SNP9, SNP10 and SNP11. More preferably, said SNP associated with obesity can be SNP7 or SNP8. In a further embodiment, said single SNPs each independently associated with obesity are also SNP9, SNP10 and SNP11.

Preferably, the alteration in the gene locus of PPY, PYY or GIP is determined by performing a hydridization assay, a sequencing assay, a microsequencing assay, an allele-specific amplification assay.

A particular aspect of this invention resides in compositions of matter comprising primers, probes, and/or oligonucleotides, which are designed to specifically detect at least one SNP or haplotype associated with obesity in the genomic region including the PPY, PYY or GIP gene, or a combination thereof. Preferably, said haplotype associated with obesity comprises SNPs selected from the group consisting of SNP4, SNP5, SNP6, SNP7, SNP8, SNP9, SNP10 and SNP11. In a preferred embodiment, said haplotype associated with obesity are comprised of SNP4, SNP5 and SNP6. Optionally, this haplotype can further comprise SNP7 and SNP8. In an alternative preferred embodiment, said haplotype associated with obesity are comprised of SNP5, SNP6 and SNP7. Optionally, this haplotype can further comprise SNP4 and/or SNP8. Preferably, said SNP associated with obesity is SNP7 or SNP8. In an other preferred embodiment, said haplotype associated with obesity are comprised of SNP9 and SNP10 or SNP10 and SNP11. Optionally, said haplotype associated with obesity are comprised of SNP9, SNP10 and SNP11. Preferably, said SNP independently associated with obesity is SNP9, SNP10 or SNP11.

Another aspect of this invention resides in binding assays utilizing the PPY and PYY gene, PPY and PYY expression products, and/or antibodies thereto. Further aspect of this invention resides in binding assays utilizing the GIP gene, GIP expression products, and/or antibodies thereto.

### LEGEND TO THE FIGURES

FIG. 1: High density mapping using Genomic Hybrid Identity Profiling (GenomeHIP) A total of 2263 BAC clones with an average spacing of 1.2 Mega base pairs between clones representing the whole human genome were tested for linkage using GenomeHIP. Each point on the x-axis corresponds to a clone. Several clones are indicated by their library name for better orientation (e.g. BACA9ZF10). Significant evidence for linkage was calculated for clone BACA9ZF10 (p-value 1.7×10⁻⁵). Suggestive evidence for linkage was calculated for clones BACA12ZA06 and BACA16ZFO2 (p-value 2.1×10⁻⁵ and 7.3×10⁻⁵, respectively). The whole linkage region is encompassing a region starting from 42214759 base pairs to 475-45876 base pairs on human chromosome 17. The p-value 2×10⁻⁵ corresponding to the significance level for significant linkage and the p-value 3×10⁴ corresponding to the significance level for suggestive linkage was used as a significance level for whole genome screens as proposed by Lander and Kruglyak (1995).

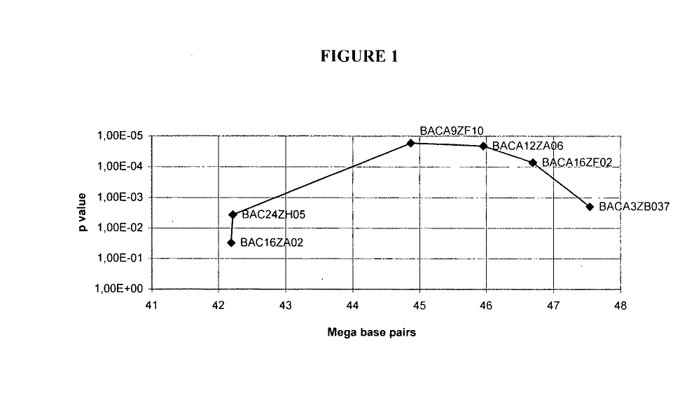

### DETAILED DESCRIPTION OF THE INVENTION

The present invention discloses the identification of PPY, PYY and GIP as human obesity susceptibility genes. Various nucleic acid samples from 164 families with obesity were submitted to a particular GenomeHIP process. This process led to the identification of particular identical-by-descent fragments in said populations that are altered in obese subjects. By screening of the IBD fragments, the inventors identified the pancreatic polypeptide (PPY) gene, the peptide YY (PYY) gene and the gastric inhibitory polypeptide (GIP) gene as candidates for obesity and related phenotypes. These genes are indeed present in the critical interval and express a functional phenotype consistent with a genetic regulation of obesity. Several SNPs located within the critical interval including the PPY, PYY and GIP genes were also identified, as being correlated to obesity in human subjects. Haplotypes comprising of SNP4, SNP5, SNP6, SNP7 and SNP8 were found to be associated with obesity. In addition, SNP7 and SNP8 were each found to be independently associated with obesity. Haplotypes comprising of SNP9, SNP10 and SNP11 were also be found to be associated with obesity and SNP9, SNP10 and SNP11 were also each independently associated with obesity. Otherwise, other haplotypes comprising of SNPs selected from the group consisting of SNP4, SNP5, SNP6, SNP7, SNP8, SNP9, SNP10 and SNP11 were also found to be associated with obesity.

The present invention thus proposes to use PPY, PYY and GIP genes for the diagnosis, and prevention of obesity and associated disorders.

### DEFINITIONS

Obesity and metabolic disorders: Obesity shall be construed as any condition of abnormal or excessive fat accumulation in adipose tissue, to the extent that health may be impaired. Associated disorders, diseases or pathologies include, more specifically, any metabolic disorders, including, but not limited to, hypo-alphalipoproteinemia, familial combined hyperlipidemia, insulin resistant syndrome X or multiple metabolic disorder, coronary artery disease, diabetes mellitus and associated complications and dyslipidemia. The invention may be used in various subjects, particularly human, including adults, children and at the prenatal stage.

Within the context of this invention, the PPY and PYY gene locus designate all PPY and PYY sequences or products in a cell or organism, including PPY and PYY coding sequences, PPY and PYY non-coding sequences (e.g., introns), PPY and PYY regulatory sequences controlling transcription translation, and/or RNA or protein stability (e.g., promoter, enhancer, terminator, etc.), as well as all corresponding expression products, such as PPY and PYY RNAs (e.g., mRNAs) and PPY and PYY polypeptides (e.g., a pre-protein and a mature protein). The PPY and PYY gene locus also comprise surrounding sequences of the PPY and PYY gene which include SNPs that are in linkage disequilibrium with SNPs located in the PPY and PYY gene. For example, the PPY and PYY locus comprises surrounding sequences comprising SNP4 to SNP8.

Within the context of this invention, the GIP gene locus designate all GIP sequences or products in a cell or organism, including GIP coding sequences, GIP non-coding sequences (e.g., introns), GIP regulatory sequences controlling transcription translation, and/or RNA or protein stability (e.g., promoter, enhancer, terminator, etc.), as well as all corresponding expression products, such as GIP RNAs (e.g., mRNAs) and GIP polypeptides (e.g., a pre-protein and a mature protein). The GIP gene locus also comprise surrounding sequences of the GIP gene which include SNPs that are in linkage disequilibrium with SNPs located in the GIP gene. For example, the GIP locus comprises surrounding sequences comprising SNP9, SNP10 and SNP11.

As used in the present application, the term “PPY gene” and the term “PYY gene” designate the human pancreatic polypeptide gene and the peptide YY gene, respectively, on human chromosome 17, as well as variants, analogs and fragments thereof, including alleles thereof (e.g., germline mutations) which are related to susceptibility to or protection from obesity and metabolic disorders. The PPY gene may also be referred to as the pancreatic icosapeptide, the PNP, or the pancreatic hormone precursor (pancreatic polypeptide) (PP) gene. The PYY gene may be referred to as the peptide YY precursor (PYY) (Peptide tyrosine tyrosine) gene.

As used in the present application, the term “GIP gene” designates the gastric inhibitory polypeptide gene on human chromosome 17, as well as variants, analogs and fragments thereof, including alleles thereof (e.g., germline mutations) which are related to susceptibility to or protection from obesity and metabolic disorders. The GIP gene may be referred to as the glucose-dependent insulinotropic polypeptide.

The term “gene” shall be construed to include any type of coding nucleic acid, including genomic DNA (gDNA), complementary DNA (cDNA), synthetic or semi-synthetic DNA, as well as any form of corresponding RNA. The term gene particularly includes recombinant nucleic acids encoding PPY, PYY or GIP, i.e., any non naturally occurring nucleic acid molecule created artificially, e.g., by assembling, cutting, ligating or amplifying sequences. A PPY, PYY and GIP gene are typically double-stranded, although other forms may be contemplated, such as single-stranded. PPY, PYY and GIP genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof. Suitable PPY gene sequences may be found on gene banks, such as Unigene Cluster for PPY (Hs.184604), Unigene Representative Sequence NM_{—}002722, REFSEQ mRNAs: NM_{—}002722, MIPS assembly: H33998S1, DOTS assembly: DT.208492, DT.100018387 and additional gene/cDNA sequences: BC032225, BC040033, M11726, M15788, X00491. Suitable PYY gene sequences may be found on gene banks, such as Unigene Cluster for PYY (Hs.169249), Unigene Representative Sequence NM_{—}004160, REFSEQ mRNAs: NM_{—}004160, MIPS assembly: H30710S1, H30710S2, DOTS assembly: DT.452118, DT.87046273 and additional gene/cDNA sequences: BC041057, D13897, D13899, D13902, L25648.1. Suitable GIP gene sequences may be found on gene banks, such as Unigene Cluster for GIP (Hs.1454), mRNAs: NM_{—}004123.

A PPY polypeptide designates any protein or polypeptide encoded by a PPY gene as disclosed above. A PYY polypeptide designates any protein or polypeptide encoded by a PYY gene as disclosed above. A GIP polypeptide designates any protein or polypeptide encoded by a GIP gene as disclosed above. The term “polypeptide” refers to any molecule comprising a stretch of amino acids. This term includes molecules of various length, such as peptides and proteins. The polypeptide may be modified, such as by glycosylations and/or acetylations and/or chemical reaction or coupling, and may contain one or several non-natural or synthetic amino acids.

A fragment of a PYY, PPY or GIP gene designates any portion of at least about 8 consecutive nucleotides of a sequence as disclosed above, preferably at least about 15, more preferably at least about 20 nucleotides, further preferably of at least 30 nucleotides. Fragments include all possible nucleotide length between 8 and 100 nucleotides, preferably between 15 and 100, more preferably between 20 and 100.

Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

### Diagnosis

The invention now provides diagnosis methods based on a monitoring of the PPY, PYY and/or GIPgene locus in a subject. Within the context of the present invention, the term “diagnosis” includes the detection, monitoring, dosing, comparison, etc., at various stages, including early, pre-symptomatic stages, and late stages, in adults, children and pre-birth. Diagnosis typically includes the prognosis, the assessment of a predisposition or risk of development or protection, the characterization of a subject to define most appropriate treatment (pharmaco-genetics), etc.

A particular object of this invention resides in a method of detecting the presence of or predisposition to obesity or an associated disorder in a subject, the method comprising (i) providing a sample from the subject and (ii) detecting the presence of an alteration in the PPY, PYY and/or GIP gene locus in said sample. In a particular embodiment, the method comprises detecting the presence of an alteration in the PPY and/or PYY gene locus in said sample. In another particular embodiment, the method comprises detecting the presence of an alteration in the GIP gene locus in said sample.

A further object of this invention resides in a method of detecting the presence of or predisposition to obesity or an associated disorder in a subject, the method comprising detecting the presence of an alteration in the PPY, PYY and/or GIP gene locus in a sample from the subject, the presence of said alteration being indicative of the presence of or the predisposition to obesity or an associated disorder. In a particular embodiment, the method comprises detecting the presence of an alteration in the PPY and/or PYY gene locus in said sample. In another particular embodiment, the method comprises detecting the presence of an alteration in the GIP gene locus in said sample.

Another particular object of this invention resides in a method of detecting the protection from obesity or an associated disorder in a subject, the method comprising detecting the presence of an alteration in the PPY, PYY and/or GIP gene locus in a sample from the subject, the presence of said alteration being indicative of the protection from obesity or an associated disorder. In a particular embodiment, the method comprises detecting the presence of an alteration in the PPY and/or PYY gene locus in said sample. In another particular embodiment, the method comprises detecting the presence of an alteration in the GIP gene locus in said sample.

In a preferred embodiment, said alteration is one or several SNP(s) or a haplotype of SNPs associated with obesity. More preferably, said haplotype associated with obesity comprises SNPs selected from the group consisting of SNP4, SNP5, SNP6, SNP7, SNP8, SNP9, SNP10 and SNP11. In a particular embodiment, said haplotype associated with obesity comprises or consists of SNP4, SNP5 and SNP6 (haplotype 4-5-6). Optionally, the haplotype 4-5-6 can further comprise SNP7 and SNP8. In an alternative embodiment, said haplotype associated with obesity comprises or consists of SNP5, SNP6 and SNP7 (haplotype 5-6-7). Optionally, the haplotype 5-6-7 can further comprise SNP4 and/or SNP8. In an additional embodiment, said haplotype associated with obesity comprises or consists of SNP9 and SNP10, or SNP10 and SNP11. Optionally, said haplotype associated with obesity comprises or consists of SNP9, SNP10 and SNP11. More preferably, said SNP associated with obesity can be selected from the group consisting of SNP7, SNP8, SNP9, SNP10 and SNP11. Still more preferably, said SNP associated with obesity can be SNP7 or SNP8. In a further embodiment, said single SNPs each independently associated with obesity are also SNP9, SNP10 and SNP11.

Another particular object of this invention resides in a method of assessing the response of a subject to a treatment of obesity or an associated disorder, the method comprising (i) providing a sample from the subject and (ii) detecting the presence of an alteration in the PPY, PYY and/or GIP gene locus in said sample. In a particular embodiment, the method comprises detecting the presence of an alteration in the PPY and/or PYY gene locus in said sample. In another particular embodiment, the method comprises detecting the presence of an alteration in the GIP gene locus in said sample.

A further object of this invention resides in a method of assessing the response of a subject to a treatment of obesity or an associated disorder in a subject, the method comprising detecting the presence of an alteration in the PPY, PYY and/or GIP gene locus in a sample from the subject, the presence of said alteration being indicative of a particular response to said treatment. In a particular embodiment, the method comprises detecting the presence of an alteration in the PPY and/or PYY gene locus in said sample. In another particular embodiment, the method comprises detecting the presence of an alteration in the GIP gene locus in said sample.

In a preferred embodiment, said alteration is one or several SNP(s) or an haplotype of SNPs associated with obesity. More preferably, said haplotype associated with obesity comprises SNPs selected from the group consisting of SNP4, SNP5, SNP6, SNP7, SNP8, SNP9, SNP10 and SNP11. In a particular embodiment, said haplotype associated with obesity comprises or consists of SNP4, SNP5 and SNP6 (haplotype 4-5-6). Optionally, the haplotype 4-5-6 can further comprise SNP7 and SNP8. In an alternative embodiment, said haplotype associated with obesity comprises or consists of SNP5, SNP6 and SNP7 (haplotype 5-6-7). Optionally, the haplotype 5-6-7 can further comprise SNP4 and/or SNP8. In an additional embodiment, said haplotype associated with obesity comprises or consists of SNP9 and SNP10, or SNP10 and SNP11. Optionally, said haplotype associated with obesity comprises or consists of SNP9, SNP10 and SNP11. More preferably, said SNP associated with obesity can be SNP7 or SNP8. In a further embodiment, said single SNPs each independently associated with obesity are also SNP9, SNP10 and SNP11.

This invention also relates to a method of determining the efficacy of a treatment of obesity or an associated disorder, the method comprising (i) providing a sample from the subject during or after said treatment, (ii) determining the PPY, PYY and/or GIP gene locus status in said sample and (iii) comparing said PPY, PYY and/or GIP gene locus status to a reference PPY, PYY and/or GIP gene locus status in a sample from said subject prior to or at an earlier stage of the treatment. In a particular embodiment, the method comprises (ii) determining the PPY and/or PYY gene locus status in said sample and (iii) comparing said PPY and/or PYY gene locus status to a reference PPY and/or PYY gene locus status in a sample from said subject prior to or at an earlier stage of the treatment. In another particular embodiment, the method comprises (ii) determining the GIP gene locus status in said sample and (iii) comparing said GIP gene locus status to a reference GIP gene locus status in a sample from said subject prior to or at an earlier stage of the treatment.

In an additional embodiment, the invention concerns a method for preventing obesity or an associated disorder in a subject, comprising detecting the presence of an alteration in the PPY, PYY and/or GIP gene locus in a sample from the subject, the presence of said alteration being indicative of the predisposition to obesity or an associated disorder; and, administering a prophylactic treatment against obesity or an associated disorder. Said prophylactic treatment can be an administration of a drug and/or a diet. In a particular embodiment, the method comprises detecting the presence of an alteration in the PPY and/or PYY gene locus in a sample from the subject. In another particular embodiment, the method comprises detecting the presence of an alteration in the GIP gene locus in a sample from the subject.

An alteration in the PPY, PYY and/or GIP gene locus may be any form of mutation(s), deletion(s), rearrangement(s) and/or insertion(s) in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion or translocation of sequences. The PPY, PYY and/or GIP gene locus alteration may result in the creation of stop codons, frameshift mutations, amino acid substitutions, particular RNA splicing or processing, product instability, truncated polypeptide production, etc. The alteration may result in the production of a PPY, PYY and/or GIP polypeptide with altered function, stability, targeting or structure. The alteration may also cause a reduction in protein expression or, alternatively, an increase in said production.

In a particular embodiment of the method according to the present invention, the alteration in the PPY, PYY and/or GIP gene locus is selected from a point mutation, a deletion and an insertion in the PPY, PYY and/or GIP gene or corresponding expression product, more preferably a point mutation and a deletion. The alteration may be determined at the level of the PPY, PYY and/or GIP gDNA, RNA or polypeptide.

In this regard, the present invention now discloses several SNPs located in the genomic region including the PPY gene, the PYY gene and the GIP gene, which are associated with obesity. The indicated nucleotide positions are based on the current sequence of Build34 obtained from NCBI. These point mutations (or single nucleotide alterations) are reported in the following Table 2:

TABLE 2

| Nucleotide position | | | | | |
|---|---|---|---|---|---|
| in genomic | | | | | |
| sequence of | SNP | dbSNP | | Position in | Sequence |
| chromosome 17 | identity | reference | Polymorphism | locus | reference |
| 42491642 | SNP4 | rs231474 | G/T | 5′ of PPY | SEQ ID |
| | | | | locus | No 1 |
| 42493636 | SNP5 | rs231473 | A/G | Intron of | SEQ ID |
| | | | | PPY locus | No 2 |
| 42498976 | SNP6 | rs151196 | A/G | 3′ of PPY | SEQ ID |
| | | | | locus and 5′ | No 3 |
| | | | | of PYY locus | |
| 42547814 | SNP7 | rs1731902 | C/T | 3′ of PYY | SEQ ID |
| | | | | locus | No 4 |
| 42561137 | SNP8 | rs186636 | C/T | 3′ of PYY | SEQ ID |
| | | | | locus | No 5 |
| 47513770 | SNP9 | rs2291725 | C/T | Coding | SEQ ID |
| | | | | region of | No 6 |
| | | | | GIP, G103S | |
| 47520914 | SNP10 | rs937301 | C/T | 3′ of GIP | SEQ ID |
| | | | | locus | No 7 |
| 47522234 | SNP11 | rs3809770 | A/G | 3′ of GIP | SEQ ID |
| | | | | locus | No 8 |

These point mutations have been detected in subjects having obesity. Haplotypes were constructed for SNP4, SNP5, SNP6, SNP7, SNP8, SNP9, SNP10 and SNP11 to determine the naturally occurring phase for each possible SNP combination. These haplotypes were then used to test for association between all the resulting haplotypes derived from combinations of the individual alleles and obesity. The results show that haplotypes comprising SNP4, SNP5 and SNP6; or SNP5, SNP6 and SNP7; or SNP5, SNP6, SNP7 and SNP8; or SNP4, SNP5, SNP6, SNP7 and SNP8; SNP9 and SNP10; or SNP10 and SNP1; or SNP9, SNP10 and SNP11 (see Table 2 above) were associated with obesity (Tables 5 and 6).

In addition, SNP7 and SNP8 were each independently tested for association with obesity. The results show that both SNPs (see Table 2 above) are each independently associated with obesity (explained in text). Similarly, SNP9, SNP10 and SNP11 are each independently associated with obesity (Table 4).

In a first variant, the method of the present invention comprises detecting the presence of an altered PPY, PYY and/or GIP gene sequence. This can be performed by sequencing all or part of the PPY, PYY and/or GIP gene, polypeptide or RNA, by selective hybridisation or by selective amplification, for instance.

A more specific embodiment comprises detecting the presence of a SNP as disclosed in Table 2 in the genomic region including the PPY, PYY and/or GIP gene sequence of a subject.

In another variant, the method comprises detecting the presence of an altered PPY, PYY and/or GIP RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the PPY, PYY and/or GIP RNA or by selective hybridisation or selective amplification of all or part of said RNA, for instance.

In a further variant, the method comprises detecting the presence of an altered PPY, PYY and/or GIP polypeptide expression. Altered PPY, PYY and/or GIP polypeptide expression includes the presence of an altered polypeptide sequence, the presence of an altered quantity of PPY, PYY and/or GIP polypeptide, the presence of an altered tissue distribution, etc. These may be detected by various techniques known in the art, including by sequencing and/or binding to specific ligands (such as antibodies), for instance.

As indicated above, various techniques known in the art may be used to detect or quantify altered PPY, PYY and/or GIP gene or RNA expression or sequence, including sequencing, hybridisation, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridisation between nucleic acids from the subject and a probe specific for wild-type or altered PPY, PYY and/or GIP gene or RNA. The probe may be in suspension or immobilized on a substrate. The probe is typically labelled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered PPY, PYY and/or GIP gene expression profile in a sample from the subject. As indicated above, this can be accomplished more preferably by sequencing, selective hybridisation and/or selective amplification of nucleic acids present in said sample.

### Sequencing:

Sequencing can be carried out using techniques well known in the art, using automatic sequencers. The sequencing may be performed on the complete PPY, PYY and/or GIP gene or, more preferably, on specific domains thereof, typically those known or suspected to carry deleterious mutations or other alterations.

### Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

In this regard, a particular object of this invention resides in a nucleic acid primer useful for amplifying sequences from the PPY gene or locus including surrounding regions. Such primers are preferably complementary to, and hybridize specifically to nucleic acid sequences in the PPY gene locus. Particular primers are able to specifically hybridise with a portion of the PPY gene locus that flank a target region of said locus, said target region being altered in certain subjects having obesity or associated disorders. Examples of such target regions are provided in Table 2 above.

Another particular object of this invention resides in a nucleic acid primer useful for amplifying sequences from the PYY gene or locus including surrounding regions. Such primers are preferably complementary to, and hybridize specifically to nucleic acid sequences in the PYY gene locus. Particular primers are able to specifically hybridise with a portion of the PYY gene locus that flank a target region of said locus, said target region being altered in certain subjects having obesity or associated disorders. Examples of such target regions are provided in Table 2 above.

Additional particular object of this invention resides in a nucleic acid primer useful for amplifying sequences from the GIP gene or locus including surrounding regions. Such primers are preferably complementary to, and hybridize specifically to nucleic acid sequences in the GIP gene locus. Particular primers are able to specifically hybridise with a portion of the GIP gene locus that flank a target region of said locus, said target region being altered in certain subjects having obesity or associated disorders. Examples of such target regions are provided in Table 2 above.

In a more specific embodiment, the invention relates to a nucleic acid primer, wherein said primer is complementary to and hybridizes specifically to a portion of a PPY coding sequence (e.g., gene or RNA), wherein said portion is altered in certain subjects having obesity or associated disorders. In this regard, particular primers of this invention are specific for altered sequences in a PPY gene or RNA. By using such primers, the detection of an amplification product indicates the presence of an alteration in the PPY gene locus. In contrast, the absence of amplification product indicates that the specific alteration is not present in the sample.

In a more specific embodiment, the invention also relates to a nucleic acid primer, wherein said primer is complementary to and hybridizes specifically to a portion of a PYY coding sequence (e.g., gene or RNA), wherein said portion is altered in certain subjects having obesity or associated disorders. In this regard, particular primers of this invention are specific for altered sequences in a PYY gene or RNA. By using such primers, the detection of an amplification product indicates the presence of an alteration in the PPY gene locus. In contrast, the absence of amplification product indicates that the specific alteration is not present in the sample.

In a more specific embodiment, the invention also relates to a nucleic acid primer, wherein said primer is complementary to and hybridizes specifically to a portion of a GIP coding sequence (e.g., gene or RNA), wherein said portion is altered in certain subjects having obesity or associated disorders. In this regard, particular primers of this invention are specific for altered sequences in a GIP gene or RNA. By using such primers, the detection of an amplification product indicates the presence of an alteration in the GIP gene locus. In contrast, the absence of amplification product indicates that the specific alteration is not present in the sample.

A further aspect of this invention includes a pair of nucleic acid primers, wherein said pair comprises a sense and a reverse primer, and wherein said sense and reverse primer specifically amplify a PPY gene or RNA or a target region thereof, said target region being altered in certain subjects having obesity or associated disorders.

A further aspect of this invention also includes a pair of nucleic acid primers, wherein said pair comprises a sense and a reverse primer, and wherein said sense and reverse primer specifically amplify a PYY gene or RNA or a target region thereof, said target region being altered in certain subjects having obesity or associated disorders.

A further aspect of this invention also includes a pair of nucleic acid primers, wherein said pair comprises a sense and a reverse primer, and wherein said sense and reverse primer specifically amplify a GIP gene or RNA or a target region thereof, said target region being altered in certain subjects having obesity or associated disorders.

Typical primers of this invention are single-stranded nucleic acid molecules of about 5 to 60 nucleotides in length, more preferably of about 8 to about 25 nucleotides in length. The sequence can be derived directly from the sequence of the PPY, PYY and/or GIP gene locus, respectively. Perfect complementarity is preferred, to ensure high specificity. However, certain mismatches may be tolerated.

### Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild-type or altered PPY gene or RNA and/or the use of a nucleic acid probe specific for wild-type or altered PYY gene or RNA and/or the use of a nucleic acid probe specific for wild-type or altered GIP gene or RNA, followed by the detection of the presence of a hybrid. The probe may be in suspension or immobilized on a substrate or support (as in nucleic acid array or chips technologies). The probe is typically labelled to facilitate detection of hybrids.

In this regard, a particular embodiment of this invention comprises contacting the sample from the subject with a nucleic acid probe specific for an altered PPY gene locus and/or contacting the sample from the subject with a nucleic acid probe specific for an altered PYY gene locus and/or contacting the sample from the subject with a nucleic acid probe specific for an altered GIP gene locus, and assessing the formation of an hybrid. In a particular, preferred embodiment, the method comprises contacting simultaneously the sample with a set of probes that are specific, respectively, for wild type PPY gene locus and for various altered forms thereof. In a particular, preferred embodiment, the method also comprises contacting simultaneously the sample with a set of probes that are specific, respectively, for wild type PYY and for various altered forms thereof. In a particular, preferred embodiment, the method also comprises contacting simultaneously the sample with a set of probes that are specific, respectively, for wild type GIP and for various altered forms thereof. In this embodiment, it is possible to detect directly the presence of various forms of alterations in the PPY, PYY and/or GIP gene locus in the sample. Also, various samples from various subjects may be treated in parallel.

A further particular object of this invention resides in a nucleic acid probe specific for a PPY, PYY and/or GIP gene or RNA. Within the context of this invention, a probe refers to a polynucleotide sequence which is complementary to and capable of specific hybridisation with a (target portion of a) PPY, PYY and/or GIP gene or RNA, and which is suitable for detecting polynucleotide polymorphisms associated with PPY, PYY and/or GIP alleles which predispose or protect to or are associated with obesity or metabolic disorders. Probes are preferably perfectly complementary to the PPY, PYY and/or GIP gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridise to a region of a PPY, PYY and/or GIP gene or RNA that carries an alteration.

A specific embodiment of this invention is a nucleic acid probe specific for an altered (e.g., a mutated) PPY, PYY and/or GIP gene or RNA, i.e., a nucleic acid probe that specifically hybridises to said altered PPY, PYY and/or GIP gene or RNA and essentially does not hybridise to a PPY, PYY and/or GIP gene or RNA lacking said alteration. Specificity indicates that hybridisation to the target sequence generates a specific signal which can be distinguished from the signal generated through non-specific hybridisation. Perfectly complementary sequences are preferred to design probes according to this invention. It should be understood, however, that certain mismatch may be tolerated, as long as the specific signal may be distinguished from non-specific hybridisation.

Particular examples of such probes are nucleic acid sequences complementary to a target portion of the genomic region including the PPY, PYY and/or GIP gene or RNA carrying a point mutation as listed in Table 2 above. More particularly, the probes can comprise a sequence selected from the group consisting of SEQ ID Nos 1-8 or a fragment thereof comprising the SNP or a complementary sequence thereof.

The sequence of the probes can be derived from the sequences of the PPY, PYY and/or GIP gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labelling, etc.

### Specific Ligand Binding

As indicated above, alteration in the PPY, PYY or GIP gene locus may also be detected by screening for alteration(s) in PPY, PYY or GIP polypeptide sequence or expression levels. In this regard, a specific embodiment of this invention comprises contacting the sample with a ligand specific for a PPY, PYY and/or GIP polypeptide and determining the formation of a complex.

Different types of ligands may be used, such as specific antibodies. In a specific embodiment, the sample is contacted with an antibody specific for a PPY, PYY or GIP polypeptide and the formation of an immune complex is determined. Various methods for detecting an immune complex can be used, such as ELISA, radio-immunoassays (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab′2, CDR regions, etc. Derivatives include single-chain antibodies, humanized antibodies, poly-functional antibodies, etc.

An antibody specific for a PPY, PYY or GIP polypeptide designates an antibody that selectively binds a PPY, PYY or GIP polypeptide, i.e., an antibody raised against a PPY, PYY or GIP polypeptide or an epitope-containing fragment thereof. Although non-specific binding towards other antigens may occur, binding to the target PPY, PYY or GIP polypeptide occurs with a higher affinity and can be reliably discriminated from non-specific binding. Preferred antibodies are specific for wild-type PPY, PYY or GIP polypeptide or for particular altered forms thereof. Preferred embodiments of this invention use antibodies specific for altered forms of PPY, PYY and/or GIP polypeptides, e.g., mutated, truncated or extended polypeptides. In particular, altered PPY, PYY or GIP polypeptides may comprise a specific domain resulting from a frameshift mutation in the coding region. Antibodies specific for said domain allow the detection of the presence of such altered polypeptides in a sample. The ligand may be used in soluble form, or coated on a surface or support.

In a specific embodiment, the method comprises contacting a sample from the subject with (a support coated with) an antibody specific for an altered form of a PPY, PYY or GIP polypeptide, and determining the presence of an immune complex. In a particular embodiment, the sample may be contacted simultaneously, or in parallel, or sequentially, with various (supports coated with) antibodies specific for different forms of a PPY, PYY or GIP polypeptide, such as a wild-type and various altered forms thereof.

The diagnosis methods can be performed in vitro, ex vivo or in vivo, preferably in vitro or ex vivo. They use a sample from the subject, to assess the status of the PPY, PYY and/or GIP gene locus. The sample may be any biological sample derived from a subject, which contains nucleic acids or polypeptides. Examples of such samples include fluids, tissues, cell samples, organs, biopsies, etc. Most preferred samples are blood, plasma, saliva, urine, seminal fluid, etc. Prenatal diagnosis may also be performed by testing foetal cells or placental cells, for instance. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered PPY, PYY and/or GIP gene locus. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered PPY, PYY or GIP polypeptide, RNA or DNA in the sample is indicative of the presence of an altered PPY, PYY or GIP gene locus in the subject, which can be correlated to the presence, predisposition or stage of progression of obesity or metabolic disorders. For example, an individual having a germline PPY, PYY and/or GIP mutation has an increased risk of developing obesity or metabolic disorders. The determination of the presence of an altered PPY, PYY and/or GIP gene locus in a subject also allows the design of appropriate therapeutic intervention, which is more effective and customized. Also, this determination at the pre-symptomatic level allows a preventive regimen to be applied.

### Linkage Disequilibirum

Once a first SNP has been identified in a genomic region of interest, more particularly in PPY, PYY and/or GIP gene locus, the practitioner of ordinary skill in the art can easily identify additional SNPs in linkage disequilibrium with this first SNP. Indeed, any SNP in linkage disequilibrium with a first SNP associated with obesity or an associated disorder will be associated with this trait. Therefore, once the association has been demonstrated between a given SNP and obesity or an associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnostic methods according to the present invention.

For example, a linkage locus of Crohn's disease has been mapped to a large region spanning 18cM on chromosome 5q31 (Rioux et al., 2000 and 2001). Using dense maps of microsatellite markers and SNPs across the entire region, strong evidence of linkage disequilibrium (LD) was found. Having found evidence of LD, the authors developed an ultra-high-density SNP map and studied a denser collection of markers selected from this map. Multilocus analyses defined a single common risk haplotype characterised by multiple SNPs that were each independently associated using TDT. These SNPs were unique to the risk haplotype and essentially identical in their information content by virtue of being in nearly complete LD with one another. The equivalent properties of these SNPs make it impossible to identify the causal mutation within this region on the basis of genetic evidence alone.

### Causal Mutation

Mutations in the PPY, PYY and/or GIP gene which are responsible for obesity or an associated disorder may be identified by comparing the sequences of the PPY, PYY and/or GIP gene from patients presenting obesity or an associated disorder and control individuals. Based on the identified association of SNPs of PPY, PYY and/or GIP and obesity or an associated disorder, the identified locus can be scanned for mutations. In a preferred embodiment, functional regions such as exons and splice sites, promoters and other regulatory regions of the PPY, PYY and/or GIP gene are scanned for mutations. Preferably, patients presenting obesity or an associated disorder carry the mutation shown to be associated with obesity or an associated disorder and control individuals do not carry the mutation or allele associated with obesity or an associated disorder. It might also be possible that patients presenting obesity or an associated disorder carry the mutation shown to be associated with obesity or an associated disorder with a higher frequency than control individuals.

The method used to detect such mutations generally comprises the following steps: amplification of a region of the PPY, PYY and/or GIP gene comprising a SNP or a group of SNPs associated with obesity or an associated disorder from DNA samples of the PPY, PYY and/or GIP gene from patients presenting obesity or an associated disorder and control individuals; sequencing of the amplified region; comparison of DNA sequences of the PPY, PYY and/or GIP gene from patients presenting obesity or an associated disorder and control individuals; determination of mutations specific to patients presenting obesity or an associated disorder.

Therefore, identification of a causal mutation in the PPY, PYY and/or GIP gene can be carried out by the skilled person without undue experimentation by using well-known methods.

For example, the causal mutations have been identified in the following examples by using routine methods.

Hugot et al. (2001) applied a positional cloning strategy to identify gene variants with susceptibly to Crohn's disease in a region of chromosome 16 previously found to be linked to susceptibility to Crohn's disease. To refine the location of the potential susceptibility locus 26 microsatellite markers were genotyped and tested for association to Crohn's disease using the transmission disequilibrium test. A borderline significant association was found between one allele of the microsatellite marker D16S136. Eleven additional SNPs were selected from surrounding regions and several SNPs showed significant association. SNP5-8 from this region were found to be present in a single exon of the NOD2/CARD15 gene and shown to be non-synonymous variants. This prompted the authors to sequence the complete coding sequence of this gene in 50 CD patients. Two additional non-synonymous mutations (SNP12 and SNP13) were found. SNP13 was most significant associated (p=6×10⁻⁶) using the pedigree transmission disequilibrium test. In another independent study, the same variant was found also by sequencing the coding region of this gene from 12 affected individuals compared to 4 controls (Ogura et al., 2001). The rare allele of SNP13 corresponded to a 1-bp insertion predicted to truncate the NOD2/CARD 15 protein. This allele was also present in normal healthy individuals, albeit with significantly lower frequency as compared to the controls.

Similarly, Lesage et al. (2002) performed a mutational analyses of CARD15 in 453 patients with CD, including 166 sporadic and 287 familial cases, 159 patients with ulcerative colitis (UC), and 103 healthy control subjects by systematic sequencing of the coding region. Of 67 sequence variations identified, 9 had an allele frequency >5% in patients with CD. Six of them were considered to be polymorphisms, and three (SNP12-R702W, SNP8-G908R, and SNP13-1007fs) were confirmed to be independently associated with susceptibility to CD. Also considered as potential disease-causing mutations (DCMs) were 27 rare additional mutations. The three main variants (R702W, G908R, and 1007fs) represented 32%, 18%, and 31%, respectively, of the total CD mutations, whereas the total of the 27 rare mutations represented 19% of DCMs. Altogether, 93% of the mutations were located in the distal third of the gene. No mutations were found to be associated with UC. In contrast, 50% of patients with CD carried at least one DCM, including 17% who had a double mutation.

Further aspects and advantages of the present invention will be disclosed in the following experimental section, which should be regarded as illustrative and not limiting the scope of the present application.

### EXAMPLES

### 1. Identification of an Obesity Susceptibility Locus on Human Chromosome 17

A. GenomeHIP Platform to Identify the Chromosome 17 Susceptibility Genes

The GenomeHIP platform was applied to allow rapid identification of two obesity susceptibility genes.

Briefly, the technology consists of forming pairs from the DNA of related individuals. Each DNA is marked with a specific label allowing its identification. Hybrids are then formed between the two DNAs. A particular process (WO00/53802) is then applied that selects all fragments identical-by-descent (IBD) from the two DNAs in a multi step procedure. The remaining IBD enriched DNA is then scored against a BAC clone derived DNA microarray that allows the positioning of the IBD fraction on a chromosome.

The application of this process over many different families results in a matrix of IBD fractions for each pair from each family. Statistical analyses then calculate the minimal IBD regions that are shared between all families tested. Significant results (p-values) are evidence for linkage of the positive region with the trait of interest (here obesity). The linked interval can be delimited by the two most distant clones showing significant p-values.

In the present study, 164 families of German origin (178 independent sib-pairs) concordant for massive obesity (as defined by a body mass index >90th % ile) were submitted to the GenomeHIP process. The resulting IBD enriched DNA fractions were then labelled with Cy5 fluorescent dyes and hybridised against a DNA array consisting of 2263 BAC clones covering the whole human genome with an average spacing of 1.2 Mega base pairs. Non-selected DNA labelled with Cy3 was used to normalise the signal values and compute ratios for each clone. Clustering of the ratio results were then performed to determine the IBD status for each clone and pair.

By applying this procedure, several BAC clones spanning an approximately 5.35 mega-base region on chromosome 17 (bases 42200000 to 47550000) were identified, that showed significant (p-value <2.2E-05) or suggestive (p-value <7.4E-04) evidence for linkage to obesity (Table 3).

Table 3: Linkage results for chromosome 17 in the region of PPY, PYY and GIP: Indicated is the region correspondent to 3 BAC clones with evidence for linkage. The start and stop positions of the clones correspond to their genomic location based on NCBI Build34 sequence respective to the start of the chromosome (p-ter).

TABLE 3

| Human | | Proportion of | | | |
|---|---|---|---|---|---|
| chromosome | Clone | informative pairs | p-value | Start | Stop |
| 17 | BACA16ZA02v | 0.99 | 3.10E−02 | | |
| 17 | BACA24ZH05v | 0.86 | 3.70E−03 | | 42214759 |
| 17 | BACA9ZF10v | 0.95 | 1.70E−05 | 44710426 | 44872488 |
| 17 | BACA12ZA06v | 0.97 | 2.10E−05 | 45770413 | 45958511 |
| 17 | BACA16ZF02v | 0.99 | 7.30E−05 | 46470465 | 46697765 |
| 17 | BACA3ZB03v | 0.42 | 2.00E−03 | 47545876 | |

B. Identification of Obesity Susceptibility Genes on Chromosome 17

By screening the aforementioned 5.35 mega-base chromosomal region, we identified the pancreatic polypeptide (PPY), peptide YY (PYY) and gastric inhibitory peptide (GIP) genes as candidates for obesity and related phenotypes. These genes are indeed present in the critical interval, with evidence for linkage delimited by the clones outlined in Table 3 above.

Hort et al. (1995) showed that the PPY gene encoding a predicted 95-amino acid sequence polypeptide (mRNA 425 bp) and the PYY gene encoding a predicted 97-amino acid sequence polypeptide (mRNA 582 bp) are located approximately 10 kb apart on 17q21.1. The peptides encoded by the two genes, pancreatic polypeptide (PP) and peptide YY (PYY), respectively, belong to the neuropeptide Y (NPY) family of peptides displaying high sequence homology. Based on sequence comparisons between the three genes it has been concluded that NPY and PYY are the result of a gene duplication event, and that a subsequent tandem duplication produced the PPY gene (Hort et al. 1995).

Multiple receptor subtypes with different affinities for these three endogenous peptides have been identified. NPY and PYY have highest affinity for the Y1, Y2, and Y5 receptors while PP is the endogenous ligand for the Y4 site (Michel et al., 1998). An alternate endogenous form of PYY, PYY(3-36), demonstrates increased affinity for the Y2 site that is thought to be primarily a presynaptic receptor found on NPY expressing neurons (Michel et al., 1998).

Multiple actions of PP and PYY in food intake have now been described in rodents as well as humans.

Peripheral administration of PP to rodents has been shown to reduce food intake (Asakawa et al., 2002). Intravenous infusion of PP caused a sustained decrease in both appetite and food intake in healthy volunteers (Batterham et al., 2003).

Batterham et al. (2002) demonstrated that peripheral injection of PYY(3-36) in rats inhibits food intake and reduces weight gain. PYY(3-36) also inhibits food intake in mice but not in Y2r-null mice, which suggests that the anorectic effect requires the Y2 receptor. In humans, infusion of normal postprandial concentrations of PYY(3-36) significantly decreased appetite and reduced food intake by 33% over 24 hours (Batterham et al., 2002). Thus, postprandial elevation of PYY(3-36) may act through the arcuate nucleus Y2R to inhibit feeding in a gut-hypothalamic pathway.

Furthermore, PYY reduces food intake by modulating appetite circuits in the hypothalamus. Batterham et al. (2003) found that obese subjects are not resistant to the anorectic effects of PYY. Endogenous PYY levels were low in obese subjects, suggesting that PYY deficiency may contribute to the pathogenesis of obesity.

Gastric inhibitory polypeptide, also known as glucose-dependent insulinotropic polypeptide (GIP), is a 42-amino acid hormone that stimulates insulin secretion in the presence of glucose. Its sequence indicates that it is a member of a family of structurally related hormones that includes secretin, glucagon, vasoactive intestinal peptide, and growth hormone-releasing factor. Takeda et al. (1987) isolated and sequenced cDNA clones encoding the human GIP precursor. The predicted amino acid sequence of the precursor indicates that GIP is derived by proteolytic processing of a 153-residue precursor, preproGIP. The GIP moiety is flanked by polypeptide segments of 51 and 60 amino acids at its amino and carboxyl termini, respectively. GIP is released from the precursor by processing at single arginine residues.

Miyawaki et al. (2002) described a novel pathway of obesity promotion via GIP. Wild-type mice fed a high-fat diet exhibited both hypersecretion of GIP and extreme visceral and subcutaneous fat deposition with insulin resistance. In contrast, mice lacking the GIP receptor (Gipr(−/−)) fed a high-fat diet were clearly protected from both the obesity and the insulin resistance. Moreover, double-homozygous mice (Gipr(−/−), Lep(ob)/Lep(ob)) generated by crossbreeding Gipr(−/−) and obese ob/ob (Lep(ob)/Lep(ob)) mice gained less weight and had lower adiposity than Lep(ob)/Lep(ob) mice. The Gipr(−/−) mice had a lower respiratory quotient and used fat as the preferred energy substrate, and were thus resistant to obesity. Therefore, GIP directly links overnutrition to obesity and it is a potential target for anti-obesity drugs.

Sirinek et al. (1986) showed that hyperinsulinism of morbid obesity and its amelioration after gastric bypass may be caused by markedly elevated levels of GIP before surgery and its reduced release after bypass.

Since GIP appears to play a role in lipid physiology and elevated levels of GIP in response to increased nutrient loads have been associated with obesity, antagonising GIP action has been proposed as a therapeutic strategy for obesity (Meier and Nauck, 2004).

Aberrant function of GIP has also been associated with type 2 diabetes. While GIP strongly stimulates insulin release in healthy humans, the peptide has almost completely lost its insulinotropic effect in patients with type 2 diabetes.

Lynn et al. (2003) presented evidence that the GIP receptor (GIPR) is controlled at normoglycemia by the fatty acid load on the islet; however, when exposed to hyperglycemic conditions, the GIPR is down-regulated, which may contribute to the decreased responsiveness to GIP that is observed in type 2 diabetes.

Due to the insulinotropic activity of GIP, there has also been a considerable increase of interest in utilising the hormone as a potential therapy for type 2 diabetes (Gault et al., 2003).

Taken together, the linkage results provided in the present application, identifying the human PPY, PYY and GIP genes in the critical interval of genetic alterations linked to obesity on chromosome 17, with the involvement of PPY and PYY in the NPY/PP signalling pathways involved in the control of food intake and energy expenditure and the involvement of GIP in overnutrition, the inventors conclude that alterations (e.g., mutations and/or polymorphisms) in the PPY, PYY and/or GIP gene or its regulatory sequences may contribute to the development of human obesity and represent a novel target for diagnosis.

### 2. Association Study

The same families that have been used for the linkage study were also used to test for association between a specific phenotype (here obesity) in question and the genetic marker allele or haplotypes containing a specific marker allele using the transmission disequilibrium test (TDT). The TDT is a powerful association test as it is insensitive to population stratification problems in the tested sample. Briefly, the segregation of alleles from heterozygous parents to their affected offspring is tested. The portion of alleles transmitted to the affected offspring compared to the non-transmitted alleles is compared to the ratio expected under random distribution. A significant excess of allele transmission over the expected value is evidence for an association of the respective allele or haplotype with the studied obesity phenotype.

The results of this analysis show that certain alleles of the GIP gene are positively associated with obesity and therefore increase the susceptibility to disease. In the tested population, for example, the allele C of SNP10 is correlated with obesity as determined by TDT (p-value=0.007). In contrast, the allele T of SNP10 is significantly under-transmitted to autistic individuals showing that this allele helps protect from the disease.

Other SNPs associated with obesity include SNP9 as shown in the examples of the transmission of the alleles to obese patients in Table 3.

Examples of the transmission and non-transmission of the alleles to obese patients are given in Table 4.

TABLE 4

| | | | Allele not | |
|---|---|---|---|---|
| | | Allele transmitted to | transmitted to | |
| SNP | Allele | obese individuals (N) | obese individuals (N) | p-value |
| 9 | C | 142 | 105 | 0.019 |
| 9 | T | 105 | 142 | 0.019 |
| 10 | C | 148 | 105 | 0.007 |
| 10 | T | 105 | 148 | 0.007 |
| 11 | A | 130 | 100 | 0.048 |
| 11 | G | 100 | 130 | 0.048 |

In addition, haplotypes were constructed for SNPs4-11 to identify the phase for all SNPs.

The results of this analysis in the tested population showed that certain haplotypes of the PPY, PYY and GIP gene are positively associated with obesity and therefore increase the susceptibility to disease. On the other hand certain haplotypes are preferentially not transmitted to obese patients which help protect from the disease. An example for a haplotype that is preferentially transmitted to obese patients is the haplotype C-A for SNP10-SNP11, p=0.004. An example for a haplotype that is preferentially not transmitted to obese patients is the haplotype T-T for SNP10-SNP11, p=0.005.

Examples of haplotypes with preferential transmission and non-transmission to obese patients are given in Table 5.

TABLE 5

| | | | | |
|---|---|---|---|---|
| | | Frequency | Frequency of | |
| | | of haplotype | haplotype not | |
| SNPs used to | Alleles | transmitted | transmitted | |
| construct | composing | to obese | to obese | |
| haplotype | haplotype | patients | patients | p-value |

| | | | | |
|---|---|---|---|---|
| 4-5-6 | G-G-G | 39.8 | 59.0 | 0.031 |
| 5-6-7 | G-G-C | 35.0 | 53.0 | 0.055 |
| 5-6-7-8 | G-G-C-C | 30.0 | 49.0 | 0.033 |
| 4-5-6-7-8 | T-A-A-T-T | 75.5 | 54.8 | 0.028 |
| 4-5-6-7-8 | G-G-G-C-C | 30.0 | 47.0 | 0.053 |
| 9-10 | T-T | 75.0 | 114.0 | 0.005 |
| 9-10 | C-C | 110.0 | 74.0 | 0.008 |
| 10-11 | C-A | 110.0 | 71.0 | 0.004 |
| 10-11 | T-G | 69.0 | 100.0 | 0.017 |
| 9-10-11 | C-C-A | 101.0 | 66.0 | 0.007 |
| 9-10-11 | T-T-G | 66.0 | 96.0 | 0.018 |

In addition, an independent case/control association study was performed including 564 individuals with extreme, early onset obesity (cases) and 328 healthy individuals (controls) to test for association between a marker allele(s), haplotype and/or genotype and obesity.

SNPs4-8 covering the chromosomal region containing the PYY and PPY gene plus the surrounding 5′ and 3′ regions were selected for genotyping in this study.

Haplotypes were constructed for SNP4, SNP5 and SNP6 to identify the phase for all SNPs using the PHASE program (version: 2.0; Stephens et al., 2001). The distribution of haplotypes was determined in patients and compared to the distribution of haplotypes in the control group.

The statistical analysis of the distribution of the haplotypes of SNP4, SNP5 and SNP6 between the obese patients and the controls revealed a correlation to obesity (p=0.02). As shown in Table 6 haplotype G-G-A (25.98% versus 23.74%) was observed with a higher frequency in the obese individuals (cases) compared to the controls while the haplotype T-A-A (52.48% versus 54.01 was under-represented in the obese patients compared to the controls.

TABLE 6

Distribution of haplotypes for SNP4, SNP5 and
SNP6 in cases and controls.

| | | | |
|---|---|---|---|
| | Haplotype | % | % |
| Haplotype # | SNP4-SNP5-SNP6 | cases | controls |

| | | | |
|---|---|---|---|
| HAPLO:1 | G-A-A | 0.02 | 0.26 |
| HAPLO:2 | G-G-A | 25.98 | 23.74 |
| HAPLO:3 | G-G-G | 20.73 | 20.02 |
| HAPLO:4 | T-A-A | 52.48 | 54.01 |
| HAPLO:5 | T-A-G | 0.77 | 1.70 |
| HAPLO:6 | T-G-A | 0.01 | 0.21 |
| HAPLO:7 | T-G-G | 0.01 | 0.06 |

The distribution of the alleles of SNP7 and SNP8 were each independently compared between the patients and controls. Based on the distribution of the alleles of each SNP in the patients and controls, odds ratios and corresponding confidence intervals were calculated to measure the extent of the association. An odds ratio of greater than 1 means that the tested genetic marker is associated with the disease and will therefore increase the susceptibility to disease. There is a negative association if the odds ratio is smaller than 1 and the tested genetic marker helps protecting from the disease.

The results of this analysis show that certain alleles of SNP7 and SNP8 are each independently associated with obesity. A higher frequency of allele T of SNP7 was observed in the patients compared to controls (0.42 versus 0.37) resulting in an odds ratio of 1.24 (95% CI:1.01-1.51, p=0.0395) for individuals who are carrying allele T compared to those who are carrying allele C. The allele T of SNP8 was also more often present in patients compared to controls (0.39 versus 0.34) resulting in an odds ratio of 1.24 (95% CI:1.02-1.52, p=0.0351) for individuals who are carrying allele T compared to those who are carrying allele C. In addition, a higher frequency of the homozygous TT genotype was observed for both SNPs in the patients compared to the controls (0.17 versus 0.13 for SNP7 and 0.14 versus 0.10 for SNP8). This resulted in an odds ratio of 1.56 (95% CI:1.01-2.40, p=0.0425) for carriers of genotype TT of SNP7 compared to carriers of genotype CC. An odds ratio of 1.60 (95% CI:1.01-2.52, p=0.0422) was determined for carriers of genotype TT of SNP8 compared to carriers of genotype CC.

The results also show that the alleles C of SNP7 and SNP8 are negatively associated with obesity. A lower frequency of allele C of SNP7 was observed in the patients compared to controls (0.58 versus 0.63) resulting in an odds ratio of 0.81 (95% CI:0.66-0.99, p=0.0395) for individuals who are carrying allele C compared to those who are carrying allele T. The allele C of SNP8 was also more often under-represented in patients compared to controls (0.61 versus 0.66) resulting in an odds ratio of 0.81 (95% CI:0.66-0.99, p=0.0351) for carriers of this allele compared to carriers of allele T. In addition, a lower frequency of the homozygous CC genotype was observed for both SNPs in the patients compared to the controls (0.33 versus 0.39 for SNP7 and 0.37 versus 0.43 for SNP8). This resulted in an odds ratio of 0.64 (95% CI:0.42-0.99, p=0.0425) for carriers of genotype CC of SNP7 compared to carriers of genotype TT. An odds ratio of 0.63 (95% CI:0.40-0.99, p=0.0423) was determined for carriers of genotype CC of SNP8 compared to carriers of genotype TT.

### REFERENCES

Asakawa A, Inui A, Yuzuriha H et al. (2003) Characterization of the effects of pancreatic polypeptide in the regulation of energy balance. Gastroenterology 124(5): 1325-1336.
Batterham R L, Cowley M A, Small C J et al. (2002) Gut hormone PYY(3-36) physiologically inhibits food intake. Nature 418(6898): 650-654.
Batterham R L, Cohen M A, Ellis S M et al. (2003) Inhibition of food intake in obese subjects by peptide YY3-36. N Engl J Med 349(10): 941-948.
Batterham R L, Le Roux C W, Cohen M A et al. (2003) Pancreatic polypeptide reduces appetite and food intake in humans. J Clin Endocrinol Metab 88(8): 3989-3992.
Chagnon Y C, Rankinen T, Snyder E E et al. (2003) The human obesity gene map: The 2002 update. Obes Res. 11 (3):313-367.
Gault V A, O'Harte F P, Flatt P R (2003) Glucose-dependent insulinotropic polypeptide (GIP): anti-diabetic and anti-obesity potential? Neuropeptides, 37(5):253-263.
Hebebrand J, Hescker H, Himmelmann G W, Schäfer H, Remschmidt H (1994) Percentiles for the body mass index based on data of the German national nutrition survey and a review of relevant factors with an influence on body weight. Aktuelle Emahrungsmedizin 19: 259-265.
Hebebrand J, Himmelmann G W, Hescker H, Schäfer H, Remschmidt H (1996) Use of percentiles for the body mass index in anorexia nervosa: diagnostic, epidemiological and therapeutic considerations. Int J Eat Dis 19: 359-369.
Hebebrand J, Hinney A, Roth H et al. (1998) Genetische Aspekte der Adipositas. In: J Wechsler (ed) Adipositas. Ex Libris Roche. Blackwell Verlag: 105-118.
Hinney A, Schmidt A, Nottebom K, et al. (1999) Several mutations in the melanocortin-4 receptor gene including a nonsense and a frameshift mutation associated with dominantly inherited obesity in humans. J. Clin Endocrinol Metab. April; 84(4):1483-1486.
Hort Y, Baker E, Sutherland G R, Shine J, Herzog H (1995) Gene duplication of the human peptide YY gene (PYY) generated the pancreatic polypeptide gene (PPY) on chromosome 17q21.1. Genomics 26(1): 77-83.
Hugot J P, Chamaillard M, Zouali H et al. (2001) Association of NOD2 leucine-rich repeat variants with susceptibility to Crohn's disease. Nature 411(6837):599-603.
Huszar D, Lynch C A, Fairchild-Huntress V, et al. (1997) Targeted disruption of the melanocortin-4 receptor results in obesity in mice. Cell 88:131-141.
Lander E and Kruglyak L (1995) Genetic dissection of complex traits: guidelines for interpreting and reporting linkage results. Nat Genet, 11 (3):241-247.
Lesage S, Zouali H, Cezard Jpet al. (2002) CARD15/NOD2 mutational analysis and genotype-phenotype correlation in 612 patients with inflammatory bowel disease. Am J Hum Genet. 70(4):845-857.
Lynn F C, Thompson S A, Pospisilik J A et al. (2003) A novel pathway for regulation of glucose-dependent insulinotropic polypeptide (GIP) receptor expression in beta cells. FASEB J, 17(1):91-93.
Meier J J, Nauck M A (2004) GIP as a potential therapeutic agent? Horm Metab Res, 36(11-12):859-866.
Michel M C, Beck-Sickinger A, Cox H et al. (1998) XVI. International Union of Pharmacology recommendations for the nomenclature of neuropeptide Y, peptide YY, and pancreatic polypeptide receptors. Pharmacol Rev, 50(1):143-150.
Miyawaki K, Yamada Y, Ban N et al. (2002) Inhibition of gastric inhibitory polypeptide signaling prevents obesity. Nat Med, 8(7):738-42.
Ogura Y, Bonen D K, Inohara N (2001) A frameshift mutation in NOD2 associated with susceptibility to Crohn's disease. Nature 411 (6837):603-606.
Rioux J D, Silverberg M S, Daly M J (2000) Genomewide search in Canadian families with inflammatory bowel disease reveals two novel susceptibility loci. Am J Hum Genet 66(6):1863-1870.
Rioux J D, Daly M J, Silverberg M S et al. (2001) Genetic variation in the 5q31 cytokine gene cluster confers susceptibility to Crohn disease. Nat Genet 29(2): 223-228.
Schneider R (1996) Relevanz und Kosten der Adipositas in Deutschland. Ernährungs-Umschau 43:369-374.
Sirinek K R, O'Dorisio T M, Hill D, McFee A S (1986) Hyperinsulinism, glucose-dependent insulinotropic polypeptide, and the enteroinsular axis in morbidly obese patients before and after gastric bypass. Surgery, 100(4):781-787.
Stephens M, Smith N J, Donnelly P (2001) A new statistical method for haplotype reconstruction from population data. Am J Hum Genet 68(4): 978-89.
Takeda J, Seino Y, Tanaka K et al. (1987) Sequence of an intestinal cDNA encoding human gastric inhibitory polypeptide precursor. Proc Natl Acad Sci, 84(20):7005-7008.
Vaisse C, Clement K, Guy-Grand B et al (1998) A frameshift mutation in human MC4R is associated with a dominant form of obesity. Nat Genet 20:113-114.
WHO (1998) Preventing and managing the global epidemic. WHO, Geneva.
Yeo G S, Farooqi I S, Aminian S, et al (1998) A frameshift mutation in MC4R associated with dominantly inherited human obesity. Nat Genet 20:111-112.
Wolf A M, Colditz G A (1996) Social and economic effects of body weight in the United States. Am J Clin Nutr 63(3 Suppl):466S-469S.
Zhang Y, Proenca R, Maffei M, Barone M, Leopold L, Friedman J M. (1994) Positional cloning of the mouse obese gene and its human homologue. Nature 372: 425-432.

1. A method of detecting the presence of or predisposition to or protection from obesity or an associated metabolic disorder in a subject, the method comprising detecting the presence of an alteration in the PPY, PYY and/or GIP gene locus in a sample from the subject, the presence of said alteration being indicative of the presence of or the predisposition to, or the protection from obesity or an associated disorder.

2. A method of assessing the response of a subject to a treatment of obesity or an associated metabolic disorder, the method comprising detecting the presence of an alteration in the PPY, PYY and/or GIP gene locus in a sample from the subject, the presence of said alteration being indicative of a particular response to said treatment.

3. A method for preventing obesity or an associated disorder in a subject, comprising detecting the presence of an alteration in the PPY, PYY and/or GIP gene locus in a sample from the subject, the presence of said alteration being indicative of the predisposition to obesity or an associated disorder; and, administering a prophylactic treatment against obesity or an associated disorder.

4. The method of claim 1, wherein said alteration in the PPY, PYY and/or GIP gene locus is selected from a mutation, a deletion and an insertion in the PPY, PYY and/or GIP gene locus.

5. The method of claim 1, wherein the presence of an alteration in the PPY, PYY and/or GIP gene locus is detected by sequencing, selective hybridisation and/or selective amplification.

6. The method of claim 1, wherein said alteration is one or several SNP(s) or an haplotype of SNPs associated with obesity.

7. The method of claim 6, wherein said haplotype associated with obesity comprises SNPs selected from the group consisting of SNP4, SNP5, SNP6, SNP7, SNP8, SNP9, SNP10 and SNP11.

8. The method of claim 7, wherein said haplotype associated with obesity comprises SNP4, SNP5 and SNP6.

9. The method of claim 7, wherein said haplotype associated with obesity comprises SNP5, SNP6 and SNP7.

10. The method of claim 9, wherein said haplotype further comprises SNP4 or SNP8 or both.

11. The method of claim 7, wherein said haplotype associated with obesity comprises SNP9 and SNP10.

12. The method of claim 7, wherein said haplotype associated with obesity comprises SNP10 and SNP11.

13. The method of claim 11, wherein said haplotype associated with obesity comprises SNP9, SNP10 and SNP11.

14. The method of claim 6, wherein said SNP associated with obesity is selected in the group consisting of SNP7, SNP8, SNP9, SNP10 and SNP11.

15. The method of claim 14, wherein said SNP associated with obesity is SNP7 or SNP8.

16. The method of claim 14, wherein said SNP associated with obesity is SNP9, SNP10 or SNP11.

17. The method of claim 1, wherein said alteration is in the PYY gene locus.

18. The method of claim 1, wherein said alteration is in the PPY gene locus.

19. The method of claim 1, wherein said alteration is in the GIP gene locus.

20. The method of claim 2, wherein said alteration in the PPY, PYY and/or GIP gene locus is selected from a mutation, a deletion and an insertion in the PPY, PYY and/or GIP gene locus.

21. The method of claim 3, wherein said alteration in the PPY, PYY and/or GIP gene locus is selected from a mutation, a deletion and an insertion in the PPY, PYY and/or GIP gene locus.

22. The method of claim 12, wherein said haplotype associated with obesity comprises SNP9, SNP10 and SNP11.
